(19) United States
(12) Patent Application Publication
(10) Pub. No.: US 2024/0108562 A1
(43) Pub. Date: Apr. 4, 2024

(54) FLAVOUR COMPOSITIONS

(71) Applicant: Givaudan SA, VERNIER (CH)

(72) Inventors: Norikazu SAJI, Chartham Canterbury Kent (GB); Nicolas COCITO ARMANINO, Baden (CH)

(21) Appl. No.: 18/275,055

(22) PCT Filed: Feb. 24, 2022
(86) PCT No.: PCT/EP2022/054597

§ 371 Date: Jul. 31, 2023

(30) Foreign Application Priority Data
Feb. 25, 2021 (GB) 2102676.0

(51) Int. Cl. A61K 8/49; A61Q 11/00

(52) U.S. Cl. CPC A61K 8/4946; A61Q 11/00; A61K 2800/92

(57) ABSTRACT

Provided are compositions possessing an enhanced flavour character.

### TECHNICAL FIELD

The present invention generally relates to flavoured compositions received orally possessing an enhanced flavour character. There is further provided a method of enhancing the flavour character of an orally received composition, in particular oral care compositions, and the use of certain compounds to enhance the flavour character in said orally received compositions.

### BACKGROUND

Oral care flavours of natural, nature-identical and synthetic origin, in particular mint-flavours, including peppermint, spearmint, and wintergreen or mixtures thereof, are popular flavours in oral care products, such as toothpaste, mouthwash and chewing gum, e.g. to cover the taste of base ingredients and to impart a signal to a consumer that the product may deliver freshness and cleanliness upon use. Mint flavours are traditionally provided by adding mint oil to the oral care composition. Since these oils are of natural origin, the amounts available and thus the price may vary from year to year. In addition, to achieve remarkable mint flavour characteristics in an end-product, higher amounts of these natural oils are required, which increases the price.

The applicant has now found that, by the addition to a flavoured, in particular mint-flavoured, oral care compositions of a compound of formula (I) as herein below defined, or a mixture thereof, the flavour perception may be enhanced, thus less flavour is required to achieve an essentially similar flavour character.

### SUMMARY

In accordance with a first aspect of the present invention there is provided a flavour formulation comprising
a) at least one compound of formula (I), a salt or solvate thereof

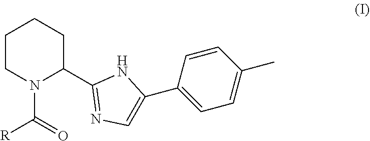

wherein R is an C₃-C₇ branched alkyl or alkenyl optionally comprising one S atom; and
b) at least one flavour, for example, a mint-flavour.

In certain embodiments, the flavour comprises at least one mint flavour selected from the list consisting of peppermint oil, spearmint oil, Mentha arvensis oil, menthol, l-carvone, l-limonene and menthone, dihydro mint lactone and mixtures thereof.

In accordance with a second aspect of the present invention there is provided an orally received composition comprising the flavour formulation of the first aspect of the invention.

In certain embodiments the orally received composition is an oral care product.

In accordance with a third aspect of the present invention there is provided a method of providing an enhanced flavour formulation, for example a mint-flavour, to a flavoured composition adapted to be received orally, comprising adding to said composition a compound of formula (I).

Certain embodiments of the present invention may provide one or more of the following advantages:
flavour loading in consumer products can be reduced without compromising on taste;
flavour reduction in turn may allow the reduction of foaming agents which are required for foaming, which is desired in particular for oral care products such as toothpaste;

### BRIEF DESCRIPTION OF THE FIGURES

FIG. 1 shows the overall flavour impression (=strength of flavour) of a flavoured toothpaste over time, flavoured with a typical a) peppermint flavour, b) spearmint flavour, and c) Wintergreen flavour.

FIG. 2 shows the perceived cooling sensitivity of a flavoured toothpaste over time, flavoured with a typical a) peppermint flavour, b) spearmint flavour, and c) Wintergreen flavour.

FIG. 3 shows the perceived freshness of a flavoured toothpaste overtime, flavoured with a typical a) peppermint flavour, b) spearmint flavour, and c) Wintergreen flavour.

In each chart a comparisons is shown over time. Further details are provided in the Examples.

### DETAILED DESCRIPTION

The present invention is based, at least in part, on the surprising finding that by addition to a flavour formulation, in particular mint-flavour formulation, of a compound of formula (I), or a mixture thereof

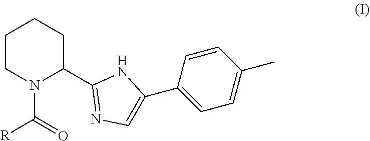

wherein R is an C₃-C₇ branched alkyl or alkenyl optionally comprising one S atom; the flavour perception may be enhanced, thus less flavour is required to achieve an essentially similar flavour character.

Thus, there is provided in a first aspect a flavour formulation comprising
a) at least one compound of formula (I), a salt or solvate thereof

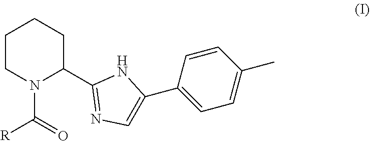

wherein R is an C₃-C₇ branched alkyl or alkenyl optionally comprising one S atom; and
b) at least one flavour, for example, a mint-flavour.

The compounds of formula (I) comprise several chiral centers and as such exist as a mixture of stereoisomers, or they may be resolved as isomerically pure forms. Resolving stereoisomers adds to the complexity of manufacture and purification of these compounds and so it is preferred to use the compounds as mixtures of their stereoisomers simply for economic reasons. However, if it is desired to prepare individual stereoisomers, this may be achieved according to methods known in the art, e.g. preparative HPLC and GC, crystallization or stereoselective synthesis. The compounds as defined by formula (I) may exist in its tautomeric forms 1H-Imidazole-3H-Imidazole form. Accordingly, the chemical structures depicted herein encompass all possible sterioisomers and tautomeric forms of the illustrated compounds.

Non-limiting examples are compounds of formula (I) wherein R is C₄-C₅ branched alkyl or alkenyl, e.g., R is but-2-yl or 2-methyl-but-3-en-2-yl.

Further non-limiting examples are compound of formula (I) wherein R is C₃-C₄ branched alkyl comprising one S atom, e.g., R is 2-methyl-3-thiabut-2-yl or 3-thiabut-2-yl.

The compounds of formula (I) selected from the group consisting of 2-methyl-1-(2-(5-(p-tolyl)-1H-imidazol-2-yl)piperidin-1-yl)butan-1-one (including (2S)-2-methyl-1-(2-(5-(p-tolyl)-1H-imidazol-2-yl)piperidin-1-yl)butan-1-one and (2R)-2-methyl-1-(2-(5-(p-tolyl)-1H-imidazol-2-yl)piperidin-1-yl)butan-1-one), 2-(methylthio)-1-(2-(5-(p-tolyl)-1H-imidazol-2-yl)piperidin-1-yl)propan-1-one, 2-methyl-2-(methylthio)-1-(2-(5-(p-tolyl)-1H-imidazol-2-yl)piperidin-1-yl)propan-1-one, and 2,2-dimethyl-1-(2-(5-(p-tolyl)-1H-imidazol-2-yl)piperidin-1-yl)but-3-en-1-one.

To achieve a noticeable effect the compound of formula (I) as defined hereinabove the addition of as little as 0.1 ppm (e.g. 0.2, 0.5, 1, 2, 4, 5, 7, 10, 12, 15, 17 ppm) based on the flavoured composition adapted to be received orally is sufficient.

In one specific embodiment the flavoured composition adapted to be received orally comprises up to 200 ppm (e.g. 180, 150,100, 75, 60, 50, 40, 30, 25, 20, 19, 18 ppm) of a compound of formula (I) as defined hereinabove, or a mixture thereof.

Thus there is provided a flavour formulation comprising
a) at least 1 ppm of at least one compound of formula (I), a salt or solvate thereof

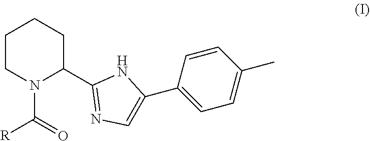

wherein R is an C₃-C₇ branched alkyl or alkenyl optionally comprising one S atom; and
b) at least one flavour, for example, a mint-flavour.

The compounds of formula (I) can be generally prepared as described in the international patent application PCT/EP2020/079009 (WO 2021/074281) which is incorporated by reference.

Generally any flavors such as those described in “Essential guide to food additives”, Third edition 2008, page 101-321 (ISBN: 978-1-905224-50-0) by Leatherhead Food International Ltd., can be used. The publication is incorporated herein by reference.

Further examples of known flavours may be found in one of the FEMA (Flavour and Extracts Manufacturers Association of the United States) publications or a compilation thereof which is available from and published by FEMA and contains all FEMA GRAS (Generally Regarded As Safe) publications from 1965 to present, eg GRAS 21 published 2003, or in Allured's Flavor and Fragrance Materials 2004, published by Allured Publishing Inc.

Examples of flavours include natural flavors, artificial flavors, spices, seasonings, and the like. Exemplary flavor ingredients include synthetic flavor oils and flavoring aromatics and/or oils, oleoresins, essences, and distillates, and a combination comprising at least one of the foregoing.

In one specific embodiment the at least one flavour is selected from anethole, menthol laevo, carvone laevo, ethyl maltol, vanillin, eucalyptol, eugenol, menthol racemic, cis-3-hexenol, linalol, mint oil (e.g. peppermint arvensis oil, peppermint piperita oil, spearmint native oil, spearmint scotch oil), corylone, ethyl butyrate, cis-3-hexenyl acetate, citral, eucalyptus oil, ethyl-vanillin, ginger oil, methyl salicylate, 2′-hydroxypropiophenone, ethyl acetate, methyl dihydro jasmonate, geraniol, lemon oil, iso amyl acetate, thymol, ionone beta, linalyl acetate, decanal, (±)-dihydromint lactone (3,6-dimethyl-3a,4,5,6,7,7a-hexahydro-3H-benzofuran-2-one), cis jasmone, ethyl hexanoate, melonal (2,6-dimethylhept-5-enal), citronellol, ethyl aceto acetate, vanilla, vanillin, nutmeg oil, rosemary oil, tea tree oil, and clove oil, or mixtures thereof.

The terms “mint flavour”, “mint-flavoured” as used herein refer to substances possessing the characteristic flavour that is a property of extracts of certain plants, notably those of the Mentha family. Examples include peppermint (Mentha piperita), spearmint (Mentha spicata), Mentha arvensis, and Mentha cardiaca, and their hybrids and fractions.

However, some other plant species can provide similar flavour, and these are also comprehended. The characteristic flavour can also be obtained or imparted by the addition of at least one of a number of compounds, non-limiting examples including l-carvone, l-limonene, menthol and menthone, the last two-named of which constitute major constituents of mint oil.

In one embodiment the mint-flavour is a mixture of two or more of natural mint oil, menthol, menthone l-carvone and l-limonene. The menthol may be either of natural origin or synthetic.

In a further embodiment, the mint-flavour is derived from compounds that do not occur naturally, but which provide the characteristic flavour. These include Frescomenthe™, ethyl vanillin, ethyl maltol, and dihydro mint lactone (CAS 92015-65-1).

Suitable natural peppermint oils include, for example, Peppermint American Far West, Peppermint American Mid West, Peppermint American Willamette, Peppermint American Yakima, Peppermint Indian piperita, and the like. Suitable spearmint oils include, for example, Spearmint American Far West Native, Spearmint American Mid West Native, Spearmint Chinese Native, Spearmint Indian Native, and the like. Suitable Mentha arvensis oils include, for example, Peppermint Chinese Arvensis, Peppermint Indian Arvensis, Peppermint Chinese Terpenless, Peppermint Indian rectified, and the like. Suitable Mentha cardiaca oils include, for example, Spearmint American Far West Scotch, Spearmint American Mid West Scotch, and the like. In addition, synthetic mint oils, such as Spearmint supra and Peppermint supra, may also be used.

It has been found, that by addition of a compound of formula (I), or a mixture thereof, to a flavour formulation, the amount of said formulation to be added to an orally received composition (e.g., an oral care composition) can be reduced by at least 10 weight % (e.g. up to 30 weight %, which includes 15, 20, and 25 weight %) based on the total amount of the orally received composition without a noticeable change of the overall flavour impression (i.e. the strength of the flavour), the cooling characteristics and/or freshness characteristics.

Similar observations have been made by reducing the amount of the mint-flavour part, e.g. menthol, in a flavour formulation. By addition of a compound of formula (I), or a mixture thereof, for example, the amount of mint-flavour, e.g. the amount of menthol, could be reduces by at least 20 weight % (e.g., up to 65 weight %, which includes 25. 35, 40, 45, 50, 55, 60 weight %) based on the flavour formulation, without a noticeable change of the overall flavour impression (i.e. the strength of the flavour), the cooling characteristics and/or freshness characteristics.

The term “orally received composition(s)” as used herein refers to food and non-food products, including oral care compositions. Oral care compositions are non-food compositions that are designed to be taken into the mouth to deliver a variety of benefits. Such compositions include dentifrices, mouthwashes, mouth sprays and gargle compositions, breath strips (edible films placed in the oral cavity to administer thereto an active agent such as a flavourant or breath-freshening agent), and chewing gums. The term “dentifrice”, as used herein, means toothpaste, oral care gels or liquids, unless otherwise specified. The dentifrice composition may be a single-phase composition or it may be a combination of two or more separate dentifrice compositions. The dentifrice composition may be in any desired form, such as deep-striped, surface-striped, multilayered, having the gel surrounding the paste, or any combination thereof.

The flavour formulations may further comprise auxiliary agents commonly used in the art, e.g. sweetener agents.

Examples of sweetening agents include, but are not limited to, sucrose, fructose, glucose, high fructose corn syrup, corn syrup, xylose, arabinose, rhamnose, erythritol, xylitol, mannitol, sorbitol, inositol, acesulfame potassium, aspartame, neotame, sucralose, and saccharine, and mixtures thereof; trilobatin, hesperetin dihydrochalcone glucoside, naringin dihydrochalcone, mogroside V, Luo Han Guo extract, rubusoside, rubus extract, glycyphyllin, isomogroside V, mogroside IV, siamenoside I, neomogroside, mukurozioside IIb, (+)-hernandulcin, 4β-hydroxyhernandulcin, baiyunoside, phlomisoside I, bryodulcoside, bryoside bryonoside, abrusosides A-E, cyclocarioside A, cyclocaryoside I, albiziasaponins A-E, glycyrrhizin, araboglycyrrhizin, periandrins I-V, pterocaryosides A and B, osladin, polypodosides A and B, telosmoside A8-18, phyllodulcin, huangqioside E neoastilbin, monatin, 3-acetoxy-5,7-dihydroxy-4′-methoxyflavanone, 2R,3R-(+)-3-Acetoxy-5,7,4′-trihydroxyflavanone, (2R,3R)-dihydroquercetin 3-O-acetate, dihydroquercetin 3-O-acetate 4′-methyl ether, brazzein, curculin, mabinlin, monellin, neoculin, pentadin, thaumatin, and combinations thereof. Some of the compounds listed above are known sweetness enhancers as well as sweeteners. When used as sweetness enhancers they are normally used below their sweetness detection thresholds.

Examples of other auxiliary agents commonly used in oral care products include, for example, surfactants, emulsifiers, solvents, colorants, preservatives, antioxidants, antimicrobial agents, enzymes, vegetal or mineral oils, fats, proteins, solubilisers, sugar derivatives, vitamins, polyols including sorbitol, organic acids, polymers, thickeners, chewing gum gum bases, and oral malodour counteracting actives. Some oral care products contain alcohols, in particular lower alcohols (C₁-C₄).

Examples of known auxiliary agents for oral care products may be found in Gaffar, Abdul, Advanced Technology, Corporate Technology, Department of Oral Care, Colgate-Palmolive Company, Piscataway, NJ, USA. Editor(s): Barel, Andre O.; Paye, Marc; Maibach, Howard I., Handbook of Cosmetic Science and Technology (2001), p.619-643. Publisher: Marcel Dekker, Inc., New York, N. Y, and in Cosmetics: Science and technology, 2nd edition, p.423-563. Edited by M. S. Balsam and E. Sagarin, Wiley Interscience, 1972.

The invention is now further described with reference to the following non-limiting examples. These examples are for the purpose of illustration only and it is understood that variations and modifications can be made by one skilled in the art.

### Example 1: Typical Flavour Formulations Suitable for Oral Care Products

1.A) Peppermint Flavour Formulation (Minty and Mentholic Fresh Profile).

| Ingredient | Parts per weight 1/100 |
|---|---|
| ANETHOLE | 12.0 |
| CLOVE Oil TERPENELESS | 0.3 |
| ETHYL MALTOL | 0.1 |
| GINGER OLEORESIN | 0.1 |
| LEMON Oil from SPAIN | 0.5 |
| L-MENTHOL | 45.0 |
| MENTHOL RACEMIC | 5.0 |
| ORANGE SWEET Oil | 0.5 |
| PEPPERMINT Oil from INDIA | 24.0 |
| PEPPERMINT Oil from NORTH AMERICA | 2.0 |
| PEPPERMINT PIPERITA Oil | 6.0 |
| PROPYLENE GLYCOL | 2.4 |
| SPEARMINT Oil | 2.0 |
| VANILLIN | 0.1 |
| | Total 100.0 |

1.B) Spearmint Flavour Formulation (Having a Ttypical Aromatic Spearmint Characteristics)

| Ingredient | Parts per weight 1/100 |
|---|---|
| ANETHOLE | 11.0 |
| L-CARVONE | 12.0 |
| ETHYL MALTOL | 0.1 |
| L-MENTHOL | 32.0 |
| PEPPERMINT Oil from INDIA | 10.0 |
| PEPPERMINT Oil from NORTH AMERICA | 6.0 |
| PROPYLENE GLYCOL | 0.8 |
| SPEARMINT Oil | 28.0 |
| VANILLA Extract | 0.1 |
| | Total 100.00 |

1.C) Wintergreen Type Flavour Formulation (Having a Typical Wintergreen Sweet Phenolic Herbal Characteristics)

| Ingredient | Parts per weight 1/100 |
|---|---|
| ANETHOLE | 11.10 |
| CLOVE Oil TERPENELESS | 1.50 |
| EUCALYPTUS Oil | 3.00 |
| EUGENOL | 7.00 |
| L-MENTHOL | 50.80 |
| METHYL SALICYLATE | 20.00 |
| PEPPERMINT Oil | 6.00 |
| PROPYLENE GLYCOL | 0.38 |
| ROSEMARY Oil | 0.04 |
| SAFRALEINE (2,3,3-trimethyl- | 0.02 |
| 2,3-dihydro-1H-inden-1-one) | |
| TEA TREE Oil | 0.10 |
| THYMOL | 0.02 |
| TOSCANOL (1-(cyclopropylmethyl)- | 0.04 |
| 4-methoxybenzene) | |
| | 100.00 |

### Example 2: Flavoured Toothpaste

Flavored toothpaste samples were prepared comprising the respective flavor formulations as indicated below.

2.A) Peppermint Flavor

“Control”: Toothpaste comprising 1.0 weight % of the peppermint flavour of Example 1.A and 0.3 weight % saccharine.

“Flavour reduction”: Toothpaste comprising 0.8 weight % the peppermint flavour of Example 1.A, 0.3 weight % saccharine, and 20 ppm of a compound of formula (I) (2-methyl-1-(2-(5-(p-tolyl)-1H-imidazol-2-yl)piperidin-1-yl)butan-1-one).

“Menthol Reduction”: Toothpaste comprising 1.0 weight % the peppermint flavour of Example 1.A, wherein 20 parts of I-menthol has been replaced by triacetin, 0.3 weight % saccharine, and 10 ppm of a compound of formula (I) (2-methyl-1-(2-(5-(p-tolyl)-1H-imidazol-2-yl)piperidin-1-yl)butan-1-one).

2.B) Spearmint Flavour

“Control”: Toothpaste comprising 0.9 weight % of the spearmint flavour of Example 1.B and 0.25 weight % saccharine.

“Flavour reduction”: Toothpaste comprising 0.7 weight % of the spearmint flavour of Example 1.B, 0.25 weight % saccharine, and 20 ppm of a compound of formula (I) (2-methyl-1-(2-(5-(p-tolyl)-1H-imidazol-2-yl)piperidin-1-yl)butan-1-one).

“Menthol Reduction”: Toothpaste comprising 0.9 weight % of the spearmint flavour of Example 1.B, wherein 20 parts of the I-menthol present in the flavour has been replaced by triacetin, 0.25 weight % saccharine, and 10 ppm of a compound of formula (I) (2-methyl-1-(2-(5-(p-tolyl)-1H-imidazol-2-yl)piperidin-1-yl)butan-1-one).

2. C) Wintergreen Type Flavor

“Control”: Toothpaste comprising 1.1 weight % of the Wintergreen type flavour of Example 1.C, and 0.3 weight % saccharine.

“Flavour reduction”: Toothpaste comprising 0.9 weight % of the Wintergreen type flavour of Example 1.C, 0.3 weight % saccharine, and 20 ppm of a compound of formula (I) (2-methyl-1-(2-(5-(p-tolyl)-1H-imidazol-2-yl)piperidin-1-yl)butan-1-one).

“Menthol Reduction”: Toothpaste comprising 1.1 weight % of the Wintergreen type flavour of Example 1.C wherein 20 parts of the I-menthol present in the flavour has been replaced by propylene glycol, 0.3 weight % saccharine, and 10 ppm of a compound of formula (I) (2-methyl-1-(2-(5-(p-tolyl)-1H-imidazol-2-yl)piperidin-1-yl)butan-1-one).

The panellists brushed with 1 gr of the toothpaste flavoured as indicated above for 1 minute before expectorating and rinsing with 10 ml water. The panellists were then asked to rate on a linear scale from 0-100 the attributes “strength”, “cooling intensity” and “freshness” immediately after brushing, and after 5, 10, 15, 20, 25, 30, 35, 40, 50, 60, 70 and 80 minutes.

The order of samples assessed was pre-determined using a balanced randomisation (Latin Square design) to minimize any carry over or order effects. The samples were assessed in a sequential monadic format.

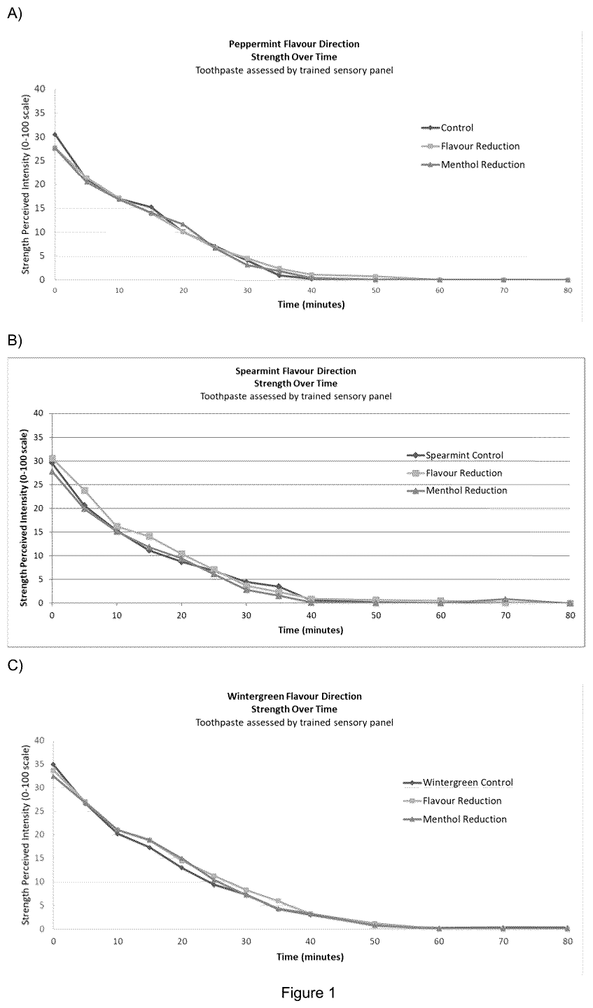

The results are shown in the charts attached (see FIGS. 1, 2 and 3).

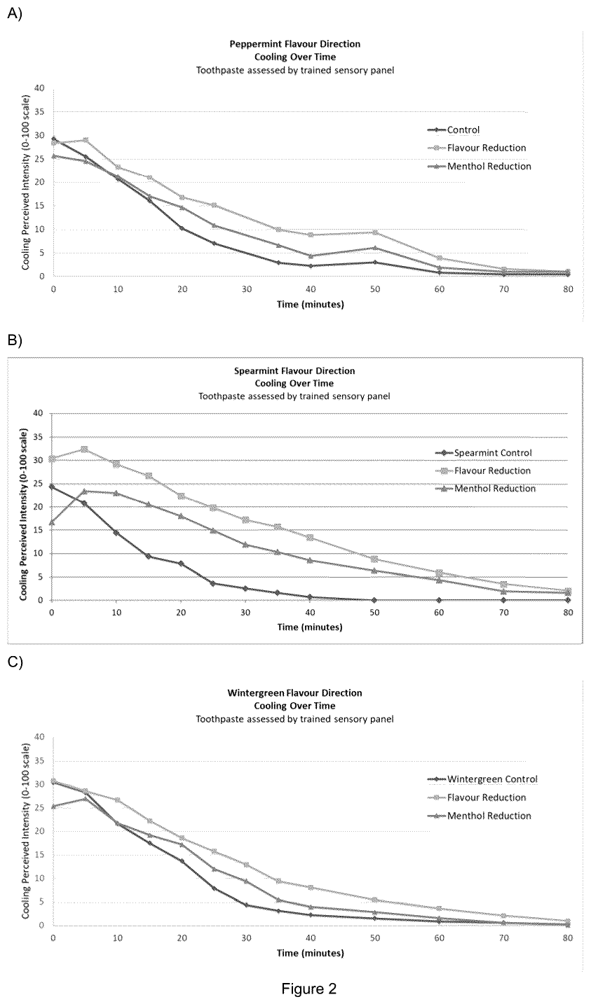

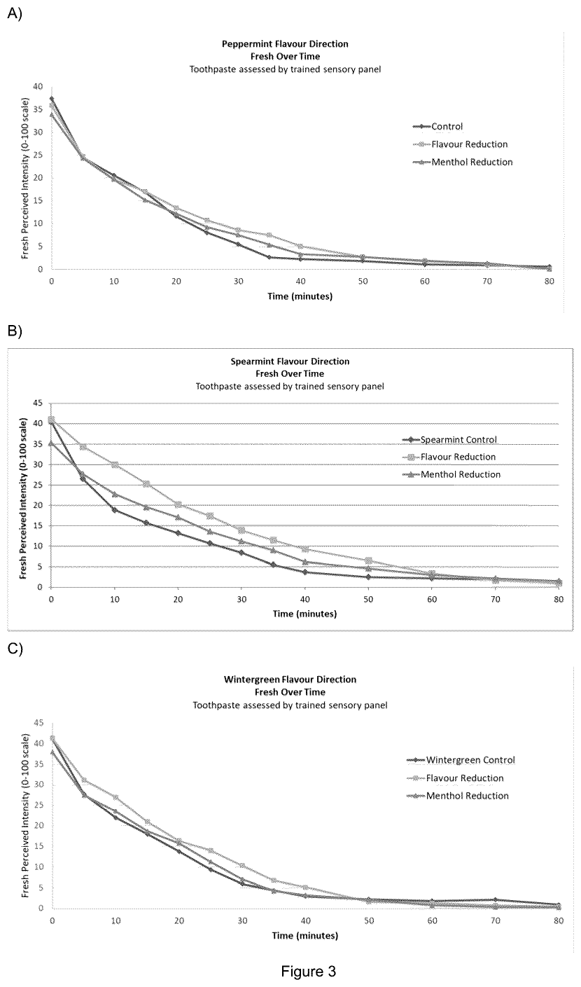

As can be seen in the charts,
the overall flavour impression (=strength of the flavour) is very similar for all samples (i.e. “control” vs “flavour reduction” vs “menthol reduction”), over time, i.e. up to 80 minutes (FIG. 1, A-C).
the perceived “cooling intensity” is substantially higher over time compared to the “control”. Both samples to which a compound of formula (I) has been added are described possessing more intense cooling properties (FIG. 2, A-C).
The perceived “freshness intensity” is similar for the Wintergreen type flavour (FIG. 3, C) and the Peppermint flavour (FIG. 3, A) and enhanced for the Spearmint flavour (FIG. 3, B).

1. A flavour formulation comprising
a) at least one compound of formula (I) 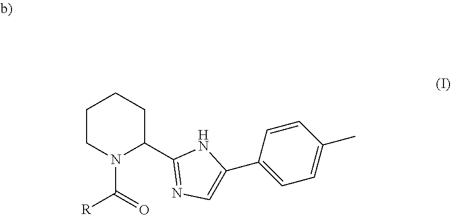
wherein R is an C₃-C₇ branched alkyl or alkenyl optionally comprising one S atom; and
c) at least one flavour.

2. The flavour formulation according to claim 1, wherein the at least one flavour is a mint flavour.

3. The flavour formulation according to claim 1, wherein the flavour is selected from the group consisting of peppermint oil, spearmint oil, Mentha arvensis oil, menthol, l-carvone, l-limonene, menthone, dihydro mint lactone and mixtures thereof.

4. The flavour formulation according to claim 3, wherein the flavour comprises at least menthol.

5. The flavour formulation according to claim 1, wherein the compound of formula (I) is selected from the group consisting of 2-methyl-1-(2-(5-(p-tolyl)-1H-imidazol-2-yl)piperidin-1-yl)butan-1-one, 2-(methylthio)-1-(2-(5-(p-tolyl)-1H-imidazol-2-yl)piperidin-1-yl)propan-1-one, 2-methyl-2-(methylthio)-1-(2-(5-(p-tolyl)-1H-imidazol-2-yl)piperidin-1-yl)propan-1-one, 2,2-dimethyl-1-(2-(5-(p-tolyl)-1H-imidazol-2-yl)piperidin-1-yl)but-3-en-1-one, and mixtures thereof.

6. An oral care composition comprising a flavour formulation as defined in claim 1.

7. A method of providing an enhanced flavour to a flavoured formulation adapted to be received orally, comprising adding to said formulation a compound of formula (I) as defined in claim 1.
